(19) United States
(12) Patent Application Publication
(10) Pub. No.: US 2024/0408144 A1
(43) Pub. Date: Dec. 12, 2024

(54) ABCB5 STEM CELL PROCESSING

(71) Applicant: Rheacell GmbH & Co., KG, Heidelberg (DE)

(72) Inventors: Jasmina Esterlechner, Heidelberg (DE); Sabrina Klingele, Heidelberg (DE); Seda Ballikaya, Heidelberg (DE); Mark Andreas Kluth, Heidelberg (DE); Maximilian Kohnle, Heidelberg (DE); Christoph Ganss, Heidelberg (DE)

(73) Assignee: Rheacell GmbH & Co., KG, Heidelberg (DE)

(21) Appl. No.: 18/699,716

(22) PCT Filed: Oct. 14, 2022
(86) PCT No.: PCT/IB2022/000617

§ 371 Date: Apr. 9, 2024

Related U.S. Application Data
(60) Provisional application No. 63/255,620, filed on Oct. 14, 2021.

(51) Int. Cl. A61K 35/28; A61J 1/06; A61J 1/10; C12N 5/0775

(52) U.S. Cl. CPC A61K 35/28; C12N 5/0662; A61J 1/06; A61J 1/10

(57) ABSTRACT

Populations of therapeutic ABCB5+ stem cells, presented in unit of use cryogenic vials are provided. Also provided are methods of making the therapeutic cells and methods of use. A closed system cryogenic vial may comprise a fill tube and an air vent connected to a cell container.

### RELATED APPLICATIONS

This application claims the benefit under 35 U.S.C. § 119(e) of U.S. provisional application No. 63/255,620, filed Oct. 14, 2021, which is incorporated by reference herein in its entirety.

### BACKGROUND OF INVENTION

Poorly defined, self-renewing adult pluripotent mesenchymal stem cells (MSCs) reside within nearly all adult connective tissues, including the dermis. Typically they maintain a niche environment, a critical requirement to protect long-term self-renewal capacity which is essential for tissue homeostasis, repair and organ maintenance.

The ATP-binding cassette sub-family B member 5, short ABCB5, also known as P-glycoprotein ABCB5 is a plasma membrane-spanning protein. The ABC superfamily of active transporters, including transporters like ABCB1 (MDR1), ABCB4 (MDR2/3) and ABCG2 (Bcrp1, MXR1) which have been suggested to be responsible for causing drug resistance in cancer patients, serves normal cellular transport, differentiation and survival functions in nonmalignant cell types. These well-known ABC transporters have been shown to be expressed at high levels on stem and progenitor cell populations. The efflux capacity for the fluorescent dyes Rhodamine123 and Hoechst 33342 mediated by these and related ABC transporters has been utilized for the isolation of such cell subsets from multiple tissues. ABCB5 identifies novel dermal and ocular cell subpopulations, for instance.

### SUMMARY OF THE INVENTION

Compositions of ABCB5⁺ stem cell populations are disclosed herein to be reliably isolated and packaged into unit of use containers in accurate therapeutic quantities.

In some aspects of the disclosure a device comprising a unit of use vial (1) comprising 10.4×10⁶±20% to 15.6×10⁶±20% ABCB5+ stem cells per ml, optionally in a volume of 1-2 ml, 1.45-1.50 ml, 4-5 ml, 1-5 ml, or 4.05-4.96 ml, wherein the unit of use vial (1) is a closed system cryogenic vial is provided.

In some aspects, a device comprising a unit of use vial (1) comprising 8×10⁶±20% to 13×10⁶±20%, 8.5×10⁶±20% to 12.5×10⁶±20%, 8.9×10⁶±20% to 12.1×10⁶±20% or 8.9×10⁶±20% to 12.4×10⁶±20% ABCB5+ stem cells per ml, after thawing, optionally in a volume of 1.5-5 ml, 1.5-4 ml, 2-5 ml, or 2-4 ml wherein the unit of use vial (1) is a closed system cryogenic vial is provided.

In some embodiments the vial (1) comprises at least one fill tube (3) connected to a cell container (6). In some embodiments the vial (1) comprises a volume of 4.50 ml. In some embodiments the vial (1) comprises a volume of 4.0 ml.

In some embodiments the vial (1) comprises 13×10⁶±20% ABCB5+ stem cells per ml.

In some embodiments the vial (1) comprises 10×10⁶±20% to 11×10⁶±20% ABCB5+ stem cells per ml, after the vial is thawed. In other embodiments the vial (1) comprises 8×10⁶ to 13×10⁶±20%, 8.5×10⁶±20% to 12.5×10⁶±20%, 8.9×10⁶±20% to 12.1×10⁶±20% or 8.9×10⁶±20% to 12.4×10⁶±20% ABCB5+ stem cells per ml, after the vial is thawed.

In some embodiments the vial (1) comprises a cell container (6), having a top end and a bottom end, wherein the top end is connected to an air vent (4) and a fill tube (3). The air vent (4) and the fill tube (3) each have a distal surface and a proximal surface, and wherein the proximal surface of each is adjacent to the top end of the cell container (6). In some embodiments the fill tube (3) connects a fill port (2) to the top end of the cell container (6). In some embodiments the fill port (2) comprises a hermetically sealed fill attachment piece for attaching a cell delivery device (16). In some embodiments the fill attachment piece is a Luer lock. In some embodiments a microbiological filter (5) is positioned within the air vent (4).

In other aspects a method for preparing a unit dose of a therapeutic cell solution is provided. The method involves preparing a pooled population comprising ABCB5-positive cells, concentrating the cells and resuspending the concentrated cells to produce a pooled sample having a cell concentration of 16×10⁶/ml-24×10⁶/ml, 13×10⁶/ml-24×10⁶/ml, 13×10⁶/ml±20%, 13×10⁶/ml±10%, 13×10⁶/ml±5%, 13×10⁶/ml±1%, or 13×10⁶/ml aliquoting a unit sample of cells from the pooled sample and transferring the unit sample to a fill tube (3) through a fill port (2) of a closed system cryogenic vial (1), and the unit sample is transferred into the cell container (6) to produce a unit dose of a therapeutic cell solution (7).

In some embodiments the method further comprises angling the vial (1) after the unit sample is transferred to the cell container (6) to expose the unit sample to fill tube (3) and allowing a sterility testing sample to pass into the fill tube (3), angling the vial (1) to an upright position and removing the sterility testing sample from the fill tube (3). In some embodiments the pooled sample has a cell concentration of about 20×10⁶/ml or about 13×10⁶/ml±10%.In some embodiments the vial (1) is angled at an about 90 degree angle.

In some embodiments the unit sample is about 5 ml.

In some embodiments the unit sample is transferred to the fill tube (3) using a Luer lock syringe (16) which is attached to the fill port (2).

In some embodiments the fill port (2) comprises a hermetically sealed fill attachment piece for attaching a cell delivery device (16). In some embodiments the sterility testing sample is removed from the fill tube (3) using a syringe.

In some embodiments the unit dose of a therapeutic cell solution (7) in the vial (1) comprises a volume of about 1.5 ml or 4.0-4.50 ml.

In some embodiments the unit dose of a therapeutic cell solution (7) in the vial (1) comprises about 13×10⁶ ABCB5+ stem cells per ml. In some embodiments the unit dose of a therapeutic cell solution (7) in the vial (1) after the cells are thawed comprises about 10×10⁶-10.5×10⁶ ABCB5+ stem cells per ml. the unit dose of a therapeutic cell solution (7) in the vial (1) after the cells are thawed comprises about 8×10⁶ to 13×10⁶, 8.5×10⁶ to 12.5×10⁶, 8.9×10⁶ to 12.1×10⁶ or 8.9×10⁶ to 12.4×10⁶ ABCB5+ stem cells per ml, after the vial is thawed.

In some embodiments the ABCB5+ stem cells population is a population of synthetic ABCB5+ stem cells, wherein greater than 96% of the population is an in vitro progeny of physiologically occurring ABCB5-positive stem cells is provided. In some embodiments greater than 96.5%, 97%, 97.5%, 98%, 98.5%, 99%, 99.5%, 99.7%, 99.9%, 99.99%, 99.998%, 99.999%, or 99.999997% of the population is an in vitro progeny of physiologically occurring ABCB5-positive stem cells. In some embodiments, 100% of the population is an in vitro progeny of physiologically occurring ABCB5-positive dermal stem cells or ocular stem cells.

Use of a population of stem cells of the invention for treating any of the disorders for which stem cell therapy is useful, tissue engineering, or wound healing using the stored cells is also provided as an aspect of the invention.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B are schematics demonstrating different embodiments of pooling of isolated ABCB5+ stem cell samples into a pooled sample which is concentrated and adjusted to optimal cell concentration.

FIGS. 2A-2B are schematics depicting different embodiments of a method for transferring a pooled sample of ABCB5+ stem cells into a closed system cryogenic vial (1), isolating an aliquot for testing, and preparing the vial for storage.

FIG. 3 is a set of diagrams depicting a method of loading cells into a closed system cryogenic vial (1). A syringe (16) is attached to an attachment piece at the top of a fill port (2) of the vial (1), cells are injected through the fill port (2) into a fill tube (3) from the syringe and allowed to pass through a filter (5) and into a cell container (6)

FIG. 4 is a diagram of a side view of a closed system cryogenic vial (1).

FIGS. 5A-5C are diagrams demonstrating an exemplary embodiments of reconstitution and delivery of thawed cells. FIG. 5A shows reconstitution solution being taken up in a delivery syringe. FIG. 5B shows that the syringe may then be used to remove the cells from the vial. FIG. 5C shows cells in the reconstitution solution may be delivered to a patient from the syringe or some may be discarded to reduce the dose before administration.

FIG. 6 is a diagram showing an alternate embodiment for reconstitution and delivery of cells.

### DESCRIPTION OF THE INVENTION

In some aspects the invention is a unit of use packaged product containing ABCB5-positive stem cells. The cells are packed in a sterile vial in an amount effective for therapeutic use. Prior to the instant invention, a method for packaging ABCB5+ stem cells for storage in a single use container were not known. A method for achieving the packaging of a critical amount of the cells, 10.4×10⁶ to 15.6×10⁶ ABCB5+ stem cells per ml, in a volume of 4.05-4.96 ml, is provided. The cells may be used for a variety of therapeutic purposes.

Thus, compositions comprised of packaged ABCB5⁺ stem cell populations are disclosed herein. These cells are isolated and packaged into unit of use containers in accurate therapeutic quantities.

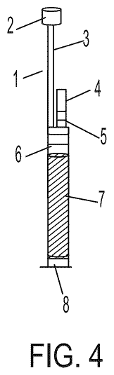

The cells are packaged in a device that comprise a unit of use vial (1), such as that shown in FIG. 4. A unit of use vial is a vial that houses a single therapeutic application of cells, that can be removed and administered directly to a patient. A vial typically is a glass or plastic container for housing a composition such as a therapeutic agent or cells, that has a closure for containing the composition or fluid content within.

The vial may have various filling capacities and may be sterile or non-sterile. Preferably the vial is sterile or sterilizable. Vials are available in a range of sizes, for housing a variety of liquid volumes, including, but not limited to 1 ml, 2 ml, 3 ml, 4 ml, 5 ml, 6 ml, 7 ml, 8 ml, 9 ml, 10 ml and greater. In some embodiments the vials used herein hold a volume of at least 5 ml. In other embodiments the vials hold a volume of 1-5 ml, 1.5-5 ml, 5-10 ml or 5-6 ml.

The vial in some embodiments is a closed system cryogenic vial. A cryogenic vial is a vial that is designed to withstand ultra-low temperatures. In some embodiments the cryogenic vial is manufactured from, for example, low binding, cryogenic grade plastics such as polypropylene, such that the vial withstands significant temperature changes. In some embodiments the vial is constructed from a commercially available cyclic olefin co-polymer (TOPAS® COC; TOPAS Advanced Polymers, GmbH). COC resins have low moisture absorption which prevents aqueous cell suspensions from adhering to the vial surface and has thermal characteristics that withstand temperatures as low at −196° C. Other materials may also be utilized.

The cryogenic process, also referred to as cryopreservation, is useful for storing viable biological systems at ultra-low temperatures in a cryogenic medium, such as liquid nitrogen, for extended periods of time. At these low temperatures, cellular metabolic activities are arrested. Cells stored under these conditions can be revived and restored to a state similar to prior to storage. When the cells are ready for use, the vial is thawed and the cells may be removed, using a device such as a syringe. The thawed cells that are removed from the vial are in a unit dose and are ready to be utilized in a therapeutic process, such as by delivery to a patient.

The quantity of in the cell container of the vial is a unit dose of a therapeutic cell solution. A unit dose of a therapeutic cell solution is an amount that is useful for delivery to a subject to produce a therapeutic result. The unit dose is defined by a concentration of cells and/or a volume. In some embodiments the unit dose of a therapeutic cell solution comprises 10.4×10⁶ to 15.6×10⁶ ABCB5+ stem cells per ml. In some embodiments, the unit dose of a therapeutic cell solution is 10.4×10⁶, 10.5×10⁶, 10.6×10⁶, 10.7×10⁶, 10.8×10⁶, 10.9×10⁶, 11.0×10⁶, 11.1×10⁶, 11.2×10⁶, 11.3×10⁶, 11.4×10⁶, 11.5×10⁶, 11.6×10⁶, 11.7×10⁶, 11.8×10⁶, 11.9×10⁶, 12.0×10⁶, 12.1×10⁶, 12.2×10⁶, 12.3×10⁶, 12.4×10⁶, 12.5×10⁶, 12.6×10⁶, 12.7×10⁶, 12.8×10⁶, 12.9×10⁶, 13.0×10⁶, 13.1×10⁶, 13.2×10⁶, 13.3×10⁶, 13.4×10⁶, 13.5×10⁶, 13.6×10⁶, 13.7×10⁶, 13.8×10⁶, 13.9×10⁶, 14.0×10⁶, 14.1×10⁶, 14.2×10⁶, 14.3×10⁶, 14.4×10⁶, 14.5×10⁶, 14.6×10⁶, 14.7×10⁶, 14.8×10⁶, 14.9×10⁶, 15.0×10⁶, 15.1×10⁶, 15.2×10⁶, 15.3×10⁶, 15.4×10⁶, 15.5×10⁶, 15.6×10⁶, 12.5×10⁶-13.5×10⁶, 12.6×10⁶-13.4×10⁶, 12.7×10⁶-13.3×10⁶, 12.8×10⁶-13.2×10⁶, or 12.9×10⁶-13.1×10⁶. In some embodiments the vial comprises 13×10⁶ ABCB5+ stem cells per ml.

In some embodiments the unit dose of a therapeutic cell solution (7) in the vial (1) after the cells are thawed comprises about 10×10⁶-10.5×10⁶ ABCB5+ stem cells per ml. In some aspects, the unit dose after the cells are thaws comprises 8×10⁶ to 13×10⁶, 8.5×10⁶ to 12.5×10⁶, 8.9×10⁶ to 12.1×10⁶ or 8.9×10⁶ to 12.4×10⁶ ABCB5+ stem cells per ml.

In some embodiments the unit dose of a therapeutic cell solution comprises a volume of 1-5 ml, 1-2 ml, 4-5 ml or 4.05-4.96 ml. In some embodiments the unit dose of a therapeutic cell solution comprises a volume of 1.50, 1.60, 1.70, 1.80. 1.90, 4.05, 4.06, 4.07, 4.08. 4.09, 4.10, 4.11, 4.12, 4.13, 4.14, 4.15, 4.16, 4.17, 4.18, 4.19, 4.20, 4.21, 4.22, 4.23, 4.24, 4.25, 4.26, 4.27, 4.28, 4.29, 4.30, 4.31, 4.32, 4.33, 4.34, 4.35, 4.36, 4.37, 4.38, 4.39, 4.40, 4.41, 4.42, 4.43, 4.44, 4.45, 4.46, 4.47, 4.48, 4.49, 4.50, 4.51, 4.52, 4.53, 4.54, 4.55, 4.56, 4.57, 4.58, 4.59, 4.60, 4.61, 4.62, 4.63, 4.64, 4.65, 4.66, 4.67, 4.68, 4.69, 4.70, 4.71, 4.72, 4.73, 4.74, 4.75, 4.76, 4.77, 4.78, 4.79, 4.80, 4.81, 4.82, 4.83, 4.84, 4.85, 4.86, 4.87, 4.88, 4.89, 4.90, 4.91, 4.92, 4.93, 4.94, 4.95, 4.96, 4.1-4.8, 4.2-4.7, 4.3-4.6, or 4.4-4.5 ml. In some embodiments the vial comprises a volume of 4.50 ml.

The vial may be any conventional design or shape. Many cryogenic vials are known in the art. In some exemplary embodiments a cryogenic vial is shown in FIG. 4. The exemplary cryogenic vial is a closed system cryogenic vial. A closed system vial is a vial that is completely sealed, for instance hermetically sealed, in particular to maintain sterility and purity of the contained cells.

The vial (1) shown in FIG. 4 comprises two main compartments, a fill tube (3) and a cell container (6). The fill tube (3) and the cell container (6) have a top end and a bottom end. The top end of the fill tube (3) may be attached to a fill port (2). The bottom end of the fill tube (3) is connected to the top end of the cell container (6). An air vent (4) is connected to the top end of the cell container (6) and a microbiological filter (5) is positioned within the air vent (4). The air vent tube (4) is also positioned adjacent to the fill tube (3) such that a bottom of the air vent tube (4) is exposed to the top end of the cell container (6) adjacent to the fill tube (3). A unit dose of a therapeutic cell solution (7) may be included in the container. A cell retrieval port (8) is positioned at the bottom of the cell container (6).

The air vent and the fill tube may be made of a more pliable material than the cell container. For instance, the fill tube and vent tube may be made of materials commonly used in cryogenic bag systems. Such materials include, for instance, ethylene vinyl acetate (EVA). In the exemplary vial shown in FIG. 4 three ports are provided, a fill port, a vent port, and a retrieval port. The fill port is designed to provide access to the cell solution, such that the cell solution may be delivered to the fill tube. It may have a needle or needle-free septum used for filling the vial. The air vent port may have a filter plug to allow air to escape, particularly as the cells are added to and fill the cell container, which displaces air. The plug may be made from polytetrafluoroethylene (PTFE) material, which is desirable as a material that can allow movement of the air under pressure and also act as a microbial barrier, thus preventing the introduction of contaminants while the cells are being added to the container or removed from the container. The fill and air vent ports are sealed, for instance, using radiofrequency (RF) or heat sealing once the cells are in the vial and the sterility sample has been removed. The retrieval port is positioned at the bottom of the cell container and is designed to allow sterile and efficient removal of the cells from the vial. Typically, the retrieval port has a needle septum that is made of pliable plastic material that may be penetrated with a needle. It may be covered prior to use to enhance sterility. Each of the ports may be hermetically sealed.

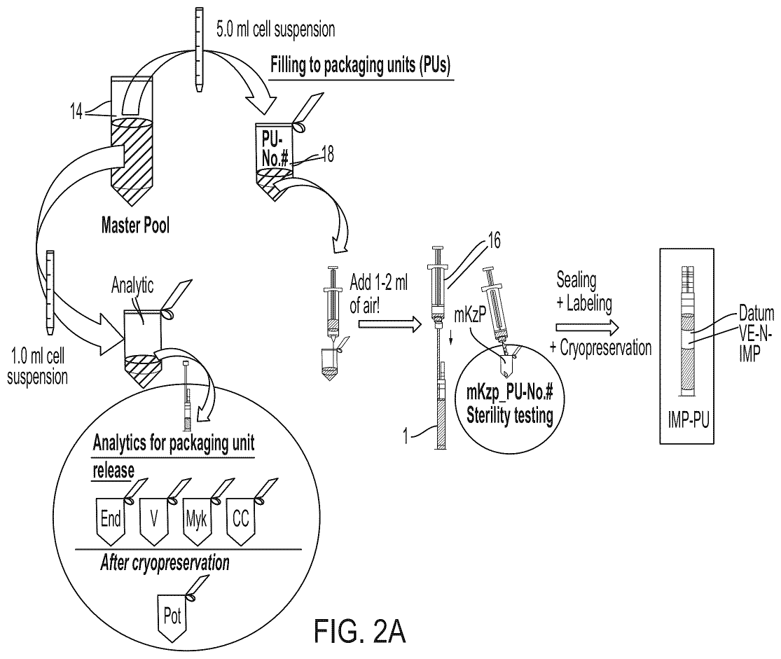

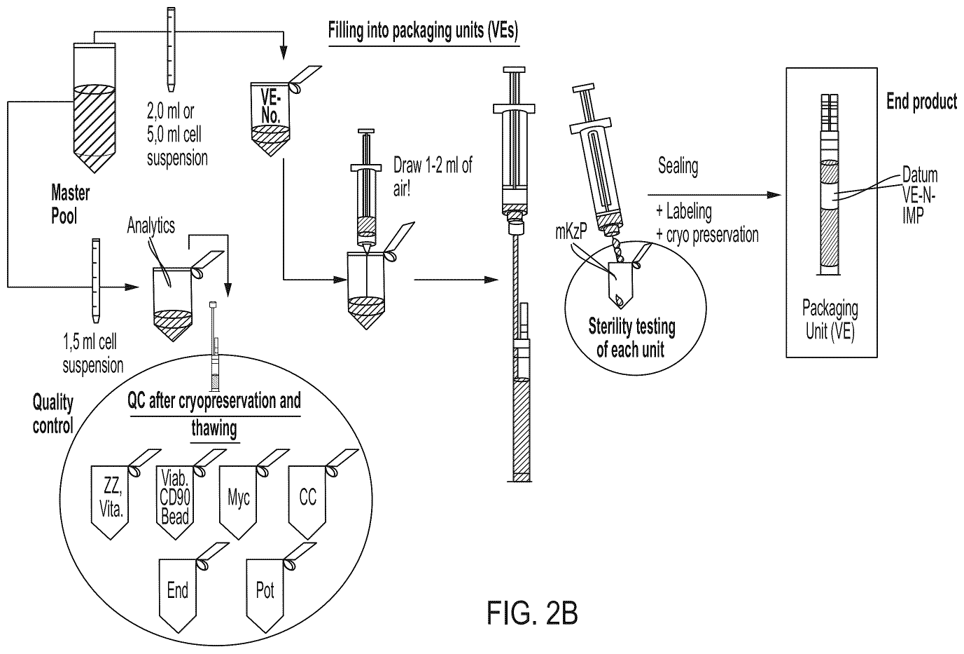

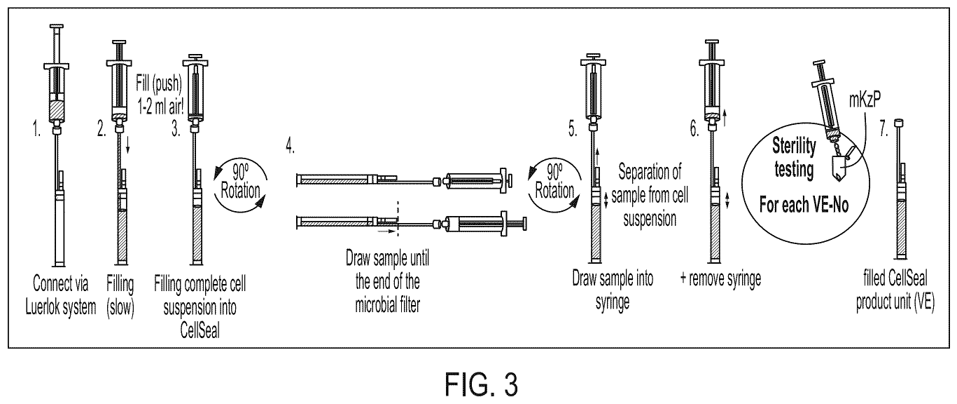

In some embodiments the fill port comprises a fill attachment piece for attaching a cell delivery device. A cell delivery device is shown for instance, as a syringe (16) in FIGS. 2 and 3. A syringe may be used to deliver the cells to the fill tube by inserting the syringe needle through the fill port and into the fill tube. In some embodiments the fill attachment piece is a Luer lock and the syringe has a luer lock tip. The luer lock has raised guides which can be fitted together with the luer lock tip of the syringe such that the syringe tip is screwed on, by being rotated to achieve a very tight fit bond between the attachment piece and the syringe. The tight fit prevents leakage and any exposure to contamination.

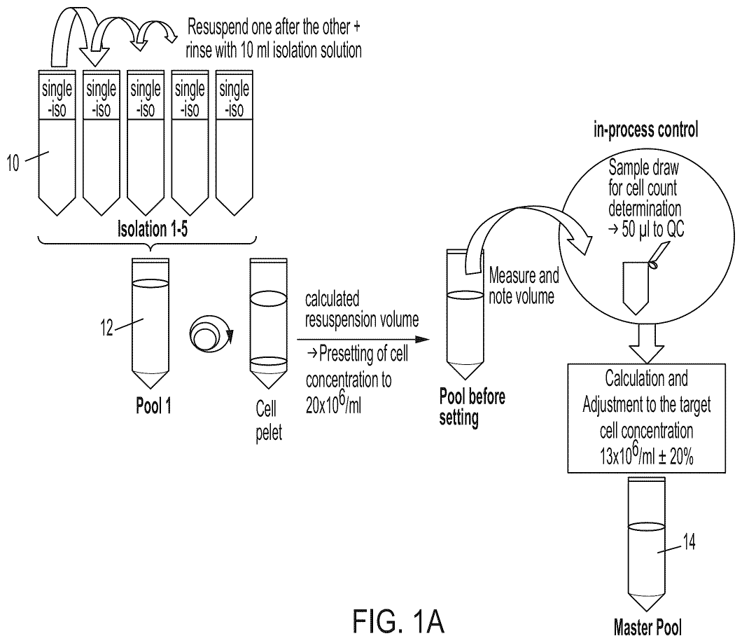

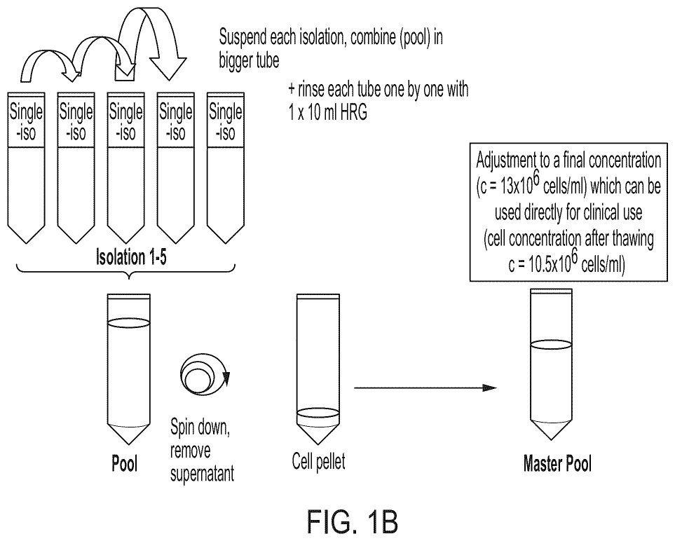

An exemplary method for preparing the unit dose of therapeutic cell solution of the invention is shown in FIGS. 1-3 and 5-6. The method initially involves preparing a pooled population comprising ABCB5-positive cells. The cells may be prepared directly from a primary source, developed as a synthetic ultra-pure cell population and/or produced by manipulating a cell population.

The ABCB5+ stem cells are isolated and transferred to tubes (10). The cells may be concentrated and resuspended in the tubes. The resuspended material is added to a pooled tube (12) to produce a pooled sample. For example, 5 single isolations containing ABCB5-positive cells may be pooled in a container such as a 50 ml tube (12). Each 50 ml tube is successively rinsed, for example with 10 ml isolation solution, to get all the remaining ABCB5+ cells out of the tube. The pooled sample of cells is then concentrated, for instance, by centrifugation. The concentrated pellet is resuspended in a given volume. In some embodiments, such as the method shown in FIG. 1A the concentration of cells is calculated once the cell pellet has been resuspended. In alternative embodiments, such as that shown in FIG. 1B the concentration of cells is calculated before the cells are pelleted, i.e. based on original cell concentrations. Following the calculations, the concentration of cells may be adjusted to produce a master pooled sample (14) having a cell concentration of 13×10⁶/ml ±20% or 16×10⁶/ml-24×10⁶/ml, or 20×10⁶/ml, which will be critical for producing the unit therapeutic dose. In some embodiments a unit dose of therapeutic cells of 13×10⁶/ml±20%. In some embodiments, the concentration of cells in the master pooled sample is about 13×10⁶/ml±20%, 13×10⁶/ml±15%, 13×10⁶/ml±10%, 13×10⁶/ml±5%, 13×10⁶/ml±3%, 13×10⁶/ml±1%, 13×10⁶/ml, 16×10⁶, 16.1×10⁶, 16.2×10⁶, 16.3×10⁶, 16.4×10⁶, 16.5×10⁶, 16.6×10⁶, 16.7×10⁶, 16.8×10⁶, 16.9×10⁶, 17.0×10⁶, 17.1×10⁶, 17.2×10⁶, 17.3×10⁶, 17.4×10⁶, 17.5×10⁶, 17.6×10⁶, 17.7×10⁶, 17.8×10⁶, 17.9×10⁶, 18.0×10⁶, 18.1×10⁶, 18.2×10⁶, 18.3×10⁶, 18.4×10⁶, 18.5×10⁶, 18.6×10⁶, 18.7×10⁶, 18.8×10⁶, 18.9×10⁶, 19.0×10⁶, 19.1×10⁶, 19.2×10⁶, 19.3×10⁶, 19.4×10⁶, 19.5×10⁶, 19.6×10⁶, 19.7×10⁶, 19.8×10⁶, 19.9×10⁶, 20.0×10⁶, 20.1×10⁶, 20.2×10⁶, 20.3×10⁶, 20.4×10⁶, 20.5×10⁶, 20.6×10⁶, 20.7×10⁶, 20.8×10⁶, 20.9×10⁶, 21.0×10⁶, 21.1×10⁶, 21.2×10⁶, 21.3×10⁶, 21.4×10⁶, 21.5×10⁶, 21.6×10⁶, 21.7×10⁶, 21.8×10⁶, 21.9×10⁶, 22.0×10⁶, 22.1×10⁶, 22.2×10⁶, 22.3×10⁶, 22.4×10⁶, 22.5×10⁶, 22.6×10⁶, 22.7×10⁶, 22.8×10⁶, 22.9×10⁶, 23.0×10⁶, 23.1×10⁶, 23.2×10⁶, 23.3×10⁶, 23.4×10⁶, 23.5×10⁶, 23.6×10⁶, 23.7×10⁶, 23.8×10⁶, 23.9×10⁶, 24.0×10⁶, or 19×10⁶-21×10⁶. In some embodiments the master pooled sample comprises about 13×10⁶ ABCB5+ stem cells per ml. In some embodiments the numbers refer to viable and/or intact cells.

It has been demonstrated that the sum of the quantity of cells in the individual cell isolates does not correspond to the total cell number after pooling due to cell loss during centrifugation. Therefore, a resuspension volume is determined in which the pooled cells are to be resuspended after centrifugation. To calculate the resuspension volume, a cell concentration of about 20×10⁶/ml is used. This adjustment in cell concentration step is essential to compensate for multiple factors in the assay in order to achieve the ultimate unit dose of cells. Higher concentrations at this step resulted in a decrease in cell viability in the end product. Additionally, the pellet volume plays a role in the final cell numbers and was, thus, utilized in setting the target concentration for the pooled sample. A target concentration of 13×10⁶/ml+/−20% for the unit cell dose was validated.

Unit samples of cells are separated from the master pooled sample for further processing. The volume of cells that are further processed and cryopreserved may vary greatly. For instance, the volume of cells that is processed and cryopreserved may be 0.5 ml-100 ml or any integer therebetween. In some embodiments the volume of cells transferred to a tube for further processing is 1 ml, 1.5 ml, 2 ml, 2.5 ml, 3 ml, 3.5 ml, 4 ml, 4.5 ml, 5 ml, 5.5 ml, 6 ml, 6.5 ml, 7 ml, 7.5 ml, 8 ml, 8.5 ml, 9 ml, 9.5 ml, 10 ml, 15 ml, 20 ml, 25 ml, 30 ml, 40 ml, or 50 ml. In one embodiment which is sued throughout, 5 ml of the cells are removed from the master pool and transferred to a syringe (16), either directly or after transfer to an individual tube (18). Although an exemplary volume of 5 ml is used throughout other volumes are contemplated and the skilled artisan can adjust cell numbers based on the volume used.

A testing sample, i.e., 1 ml of the master pooled sample may be separated and used for analytical processing. Testing samples for analytical processing may be removed from the sample before and/or after cryopreservation. The remaining cell suspension is used for the production of packaging units. For this purpose, 5 ml of cell suspension is transferred into a 5 ml tube (18) and carefully and slowly aspirated using a syringe (16) (i.e., 5 ml) and cannula, as shown in FIGS. 2A-2B. 1-2 ml air is added into the syringe to enhance the process. In preferred embodiments, a volume of 5 ml of cell suspension per packaging unit is validated, such that after filling, cryopreservation, thawing and collection, a volume of at least 4.0-4.5 ml±10% in the final product is provided. The unit sample of cells, once in the syringe (16) may be transferred to the vial (1). Specifically, after removing the cannula, the syringe is connected to the top, ie by luer lock of the cryogenic vial. The cell suspension is slowly transferred into the fill tube (3) through a fill port (2) of a closed system cryogenic vial. 1-2 ml of air is drawn into the syringe to ensure that the entire cell suspension is in the vial.

In another embodiment, as shown in FIG. 2B, 2 ml of cell suspension is transferred into a 2 ml tube (18) and carefully and slowly aspirated using a syringe (16) (i.e., 2 ml) and cannula. 1-2 ml air is added into the syringe to enhance the process. In preferred embodiments, a volume of 2 ml of cell suspension per packaging unit is validated, such that after filling, cryopreservation, thawing and collection, a volume of at least 1.5 ml±10% in the final product is provided.

After the unit sample is transferred into the fill tube it traverses from the fill tube (3) into the cell container (6). The method may further involve a step of angling the vial (1) after the unit sample is transferred to the cell container (6) to remove a small portion of the unit sample for sterility testing into the fill tube (3).

An about 900 rotation is used such that the vial is half-way inverted and the cell container (6) is facing sideways and allowing a portion of the cells to transfer back into the fill tube. After a small sample, about 0.95-0.05 ml, preferably about 0.50 ml is transferred to the fill tube, the vial is again rotated 900 such that the vial is right-side up. Two liquid phases are now presented separately. The sterility sample, which is in the fill tube is then drawn into the syringe. The Luer connection is carefully disconnected, and the syringe is removed from the vial. The sterility sample may be used for sterility testing. The fill tube and air vent tubes are then sealed. The sealed vial may then be labeled and cryopreserved.

Cryopreservation is a method that allows biological materials to be stored at very low temperatures, typically from about −80° C. to −196° C., e.g. in mechanical deep freezers or liquid nitrogen cryogenic freezers or tanks. Cryopreservation is known to store biological materials, such as cells, for a relatively long period of time, potentially indefinitely, with no functional degradation or deterioration of the biological materials. In some embodiments, the cells produced as described herein are cryopreserved and stored in the gas-phase of liquid nitrogen (<−130° C.).

Once the frozen cells are ready for use, the vial may be retrieved and thawed. A cell retrieval port as the bottom of the cell container may be accessed with a syringe to remove the cells, which are then ready for therapeutic use. The packaging unit includes the finished drug product, a unit dose of a therapeutic cell solution (7). A single unit dose of cells may be delivered directly to the patient or reconstituted to create a custom dose for a patient. Depending on the type of indication being treated and/or the size of the patient a dose may comprise a single or multiple unit doses of the therapeutic cell solution. For instance, a therapeutic quantity may range between 5.8×10⁷ and 1×10¹⁰ cells, 1×10⁷ and 1×10¹⁰, or 1×10⁷ and 2×10⁸ or greater and dosing may be repeated at regular intervals (e.g., weekly, monthly etc.) as determined to be appropriate for the particular indication and individual.

In some embodiments the unit dose in the thawed vial is 10.5×10⁶ cells/ml. In some embodiments, the concentration of cells in the thawed vial is 10×10⁶-11×10⁶. In some embodiments, the concentration of cells in the thawed vial is 10.2×10⁶-10.8×10⁶. In some embodiments, the concentration of cells in the thawed vial is 10.4×10⁶-10.6×10⁶. In some embodiments the unit dose in the thawed vial is 8×10⁶ to 13×10⁶. In some embodiments the unit dose in the thawed vial is 8.5×10⁶ to 12.5×10⁶. In some embodiments the unit dose in the thawed vial is 8.9×10⁶ to 12.1×10⁶. In some embodiments the unit dose in the thawed vial is 8.9×10⁶ to 12.4×10⁶.

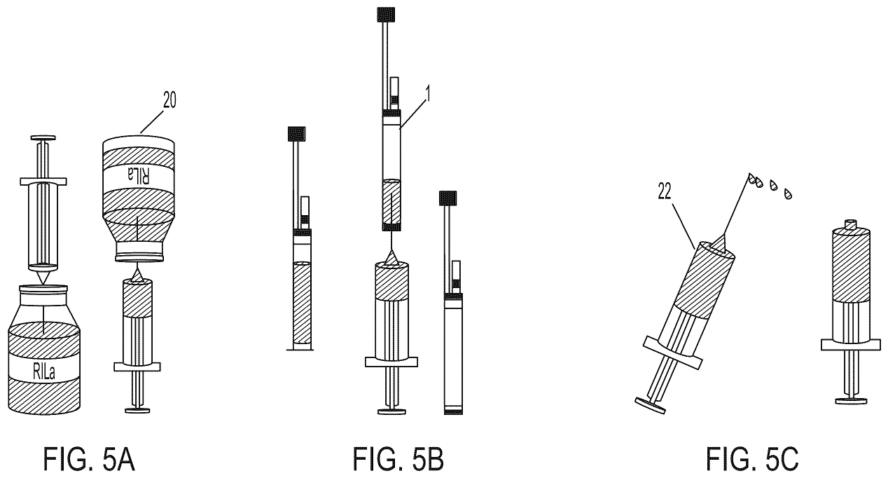

The thawed cells may be reconstituted and prepared for delivery to a patient. In some embodiments the thawed cells are reconstituted and delivered as shown in FIG. 5. A volume of reconstitution solution (20) may be taken up in, for example, a delivery syringe (22) as shown in FIG. 5A. The syringe may then be used to remove the cells from the vial (1) as shown in FIG. 5B. The total cells in the reconstitution solution may be delivered to a patient from the syringe as shown in FIG. 5C or some may be discarded to reduce the dose before administration.

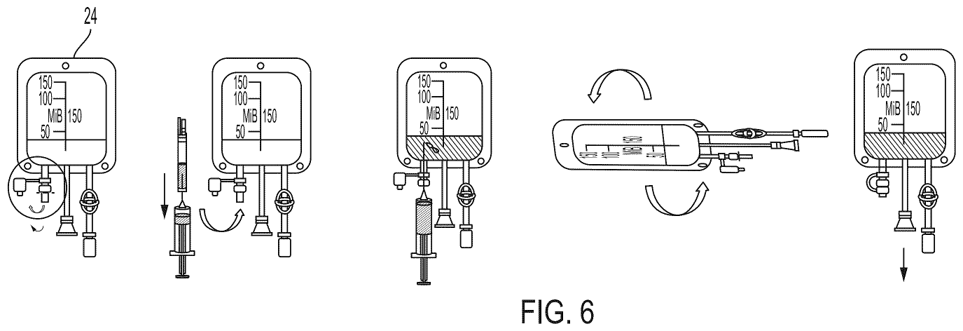

In other embodiments the cells may be reconstituted and delivered as shown in FIG. 6. Infusion bags (24) may be filled or prefilled with reconstitution solution for immediate or later reconstitution and application. After thawing of the cell vials, the cell suspension may be drawn into a syringe and transferred into the infusion bag (24). The cells may be mixed with the solution in the infusion bag before administration to a patient. The cells can be administered directly to a patient from the infusion bag.

The cells stored in the devices disclosed herein are populations of ABCB5+ stem cells. The term “population of cells” as used herein refers to a composition comprising at least two, e.g., two or more, e.g., more than one, ABCB5+ stem cells, and does not denote any level of purity or the presence or absence of other cell types, unless otherwise specified.

In an exemplary embodiment, the population is substantially free of other cell types. In another exemplary embodiment, the population comprises at least two cells of the specified cell type, or having the specified function or property.

The ABCB5+ stem cells may be isolated cells. The cells may be isolated from tissue such as skin or eye tissue, for instance, human tissue. The isolated tissue may be used directly or may be cultured.

The cell populations may in some embodiments be highly pure synthetic cell populations. In some preferred embodiments, 100% of the cells are synthetic, with 0% of the cells originating from a donor tissue such as a human tissue. In some embodiments the cell population comprises at least 95%, 95.1%, 95.2%, 95.3%, 95.4%, 95.5%, 95.6%, 95.7%, 95.8%. 95.9%, 96.0%, 96.1%, 96.2%, 96.3%, 96.4%, 96.5%, 96.6%, 96.7%, 96.8%. 96.9%, 97.0%, 97.1%, 97.2%, 97.3%, 97.4%, 97.5%, 97.6%, 97.7%, 97.8%. 97.9%, 98.0%, 98.1%, 98.2%, 98.3%, 98.4%, 98.5%, 98.6%, 98.7%, 98.8%. 98.9%, 99.0%, 99.1%, 99.2%, 99.3%, 99.4%, 99.5%, 99.6%, 99.7%, 99.8%, 99.85%, 99.86%, 99.87%, 99.88%, 99.89%, 99.90%, 99.95%, 99.96%, 99.97%, 99.98%, 99.99%, or 99.99% to 99.999997% of in vitro manufactured or synthetic ABCB5+ stem cells.

The ABCB5+ stem cells may be isolated and processed using methods known in the art. For instance, the cells may be isolated from tissue, such as skin or ocular tissue, using, for instance, ABCB5-specific antibodies. Multiple batches of ABCB5+ stem cells may be grown and passaged and then pooled to form the pooled sample. A pooled sample is a sample in which multiple single batches of ABCB5− positive cells (i.e., isolated, cultured, or synthetic cells) are combined into one. Typically, in some embodiments, one pooled sample (also referred to as a master batch) is comprised of multiple single batches that originate from the same starting material (i.e., same donor) or are isolated in parallel on the same day with the same passage number, for example.

The amount of cells in a sample and cell viability may be measured using methods known in the art. Automated methods for the determination of cell count and cell vitality include Flow Cytometry. Flow Cytometry (i.e., BD Accuri C6 Flow Cytometer) and provide a rapid and reliable method to quantify live cells in a cell suspension. One method to assess cell vitality is using dye exclusion. Live cells have intact membranes that exclude a variety of dyes that easily penetrate the damaged, permeable membranes of non-viable cells.

The determination of the cell counts as well as vitality is performed after the isolation of synthetic stem cells, directly before their cryopreservation.

An exemplary cell analysis involves pipetting 10 μl cell suspension from the single or pooled samples or the cryogenic vial into 1.5 ml reaction tubes (containing 80 μl Versene). After addition of 10 μl PI solution (1 mg/ml) the total volume is adjusted to 500 μl with Versene and the measurement is performed with the BD Accuri C6 Flow Cytometer. Cell count and vitality are calculated. An ideal acceptance criterion for cell vitality is ≥90%.

Cell viability may also be assessed using flow cytometry with a Calcein-AM (Calcein Acetoxymethylester) stain. Calcein AM is a non-fluorescent, hydrophobic compound that easily permeates intact, live cells. Upon entering the cell, intracellular esterases cleave the acetoxymethyl (AM) ester group producing calcein, a hydrophilic, strongly fluorescent compound that is well-retained in the cell cytoplasm. Apoptotic and dead cells with compromised cell membranes do not retain Calcein. Calcein is optimally excited at 495 nm and has a peak emission of 515 nm.

The cell viability measurement may be performed immediately prior to cryopreservation of the cells in order to provide information on the cell viability rate. The cell viability rate provides information about the actual metabolic activity of the isolated cells unlike the cell vitality determination with PI which only determines whether a cell is alive or dead.

The ABCB5+ stem cells are preferably isolated. An “isolated synthetic ABCB5+ stem cell” as used herein refers to a preparation of cells that are placed into conditions other than their natural environment. The term “isolated” does not preclude the later use of these cells thereafter in combinations or mixtures with other cells or in an in vivo environment and includes primary cells isolated from donors, cultured cells and synthetic cells.

The therapeutic ABCB5+ stem cells may be prepared as substantially pure preparations. The term “substantially pure” means that a preparation is substantially free of cells other than ABCB5 positive stem cells. For example, the ABCB5 cells should constitute at least 70 percent of the total cells present with greater percentages, e.g., at least 85, 90, 95 or 99 percent, being preferred.

The therapeutic ABCB5+ stem cells of the invention may be used for many different therapeutic purposes. For instance, the therapeutic cells may be used for tissue repair and regeneration, syngeneic transplants cutaneous wound healing, allogeneic transplants, peripheral arterial occlusive disease—PAOD, acute-on-chronic liver failure—AOCLF, epidermolysis bullosa—EB and many other diseases. For instance, KRT12+ corneal differentiation capacity, for treatment of limbal stem cell deficiency (LSCD) and other corneal and ocular disorders. Due to their capacity to engraft and release wound healing promoting factors, the ABCB5+ stem cells are useful for treating acute and chronic wounds.

The therapeutic ABCB5+ stem cells are useful in some embodiments for treating immune mediated diseases. Immune mediated diseases are diseases associated with a detrimental immune response, i.e., one that damages tissue. These diseases include but are not limited to transplantation, autoimmune disease, cardiovascular disease, liver disease, kidney disease and neurodegenerative disease.

It has been discovered that therapeutic ABCB5+ stem cells can be used in transplantation to ameliorate a response by the immune system such that an immune response to an antigen(s) will be reduced or eliminated. Transplantation is the act or process of transplanting a tissue or an organ from one body or body part to another. The therapeutic ABCB5+ stem cells may be autologous to the host (obtained from the same host) or non-autologous such as cells that are allogeneic or syngeneic to the host. Non-autologous cells are derived from someone other than the patient or the donor of the organ. Alternatively the therapeutic ABCB5+ stem cells can be obtained from a source that is xenogeneic to the host. In some embodiments the cells are synthetic. Thus, the therapeutic ABCB5+ stem cells are used to suppress or ameliorate an immune response to a transplant (tissue, organ, cells, etc.) by administering to the transplant recipient therapeutic ABCB5+ stem cells in an amount effective to suppress or ameliorate an immune response against the transplant.

The therapeutic ABCB5+ stem cells of the invention are also useful for treating and preventing autoimmune disease. Autoimmune disease is a class of diseases in which a subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus. A “self-antigen” as used herein refers to an antigen of a normal host tissue. Normal host tissue does not include cancer cells.

An example of autoimmune disease is anti-glomerular basement membrane (GBM) disease. GBM disease results from an autoimmune response directed against the noncollagenous domain 1 of the 3 chain of type IV collagen (3(IV)NC1) and causes a rapidly progressive glomerulonephritis (GN) and ultimately renal failure in afflicted patients. Another autoimmune disease is Crohn's disease. Clinical trials for the treatment of Crohn's disease using synthetic ABCB5+ stem cells have been conducted. Crohn's disease is a chronic condition associated with inflammation of the bowels and gastrointestinal tract.

When used in the treatment of an autoimmune disease, the therapeutic ABCB5+ stem cells will preferably be administered by intravenous injection and an effective dose will be the amount needed to slow disease progression or alleviate one or more symptoms associated with the disease. For example, in the case of relapsing multiple sclerosis, an effective dose should be at least the amount needed to reduce the frequency or severity of attacks. In the case of rheumatoid arthritis, an effective amount would be at least the number of cells needed to reduce the pain and inflammation experienced by patients.

The therapeutic ABCB5+ stem cells are also useful in the treatment of liver disease. Liver disease includes disease such as hepatitis which result in damage to liver tissue. More generally, the therapeutic ABCB5+ stem cells of the present invention can be used for the treatment of hepatic diseases, disorders or conditions including but not limited to: alcoholic liver disease, hepatitis (A, B, C, D, etc.), focal liver lesions, primary hepatocellular carcinoma, large cystic lesions of the liver, focal nodular hyperplasia granulomatous liver disease, hepatic granulomas, hemochromatosis such as hereditary hemochromatosis, iron overload syndromes, acute fatty liver, hyperemesis gravidarum, intercurrent liver disease during pregnancy, intrahepatic cholestasis, liver failure, fulminant hepatic failure, jaundice or asymptomatic hyperbilirubinemia, injury to hepatocytes, Crigler-Najjar syndrome, Wilson's disease, alpha-1-antitrypsin deficiency, Gilbert's syndrome, hyperbilirubinemia, nonalcoholic steatohepatitis, porphyrias, noncirrhotic portal hypertension, noncirrhotic portal hypertension, portal fibrosis, schistosomiasis, primary biliary cirrhosis, Budd-Chiari syndrom, hepatic veno-occlusive disease following bone marrow transplantation, etc.

In some embodiments, the invention is directed to treating a neurodegenerative disease, with ABCB5+ stem cells. In some cases, the invention contemplates the treatment of subjects having neurodegenerative disease, or an injury to nerve cells which may lead to neurodegeneration. “Neurodegenerative disorder” or “neurodegenerative disease” is defined herein as a disorder in which progressive loss of neurons occurs either in the peripheral nervous system or in the central nervous system. Non-limiting examples of neurodegenerative disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapie, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease, vertigo as result of CNS degeneration; pathologies arising with chronic alcohol or drug abuse including, for example, the degeneration of neurons in locus coeruleus and cerebellum; pathologies arising with aging including degeneration of cerebellar neurons and cortical neurons leading to cognitive and motor impairments; and pathologies arising with chronic amphetamine abuse including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor), and Wernicke-Korsakoff's related dementia. Neurodegenerative diseases affecting sensory neurons include Friedreich's ataxia, diabetes, peripheral neuropathy, and retinal neuronal degeneration. Neurodegenerative diseases of limbic and cortical systems include cerebral amyloidosis, Pick's atrophy, and Retts syndrome. The foregoing examples are not meant to be comprehensive but serve merely as an illustration of the term “neurodegenerative disorder or “neurodegenerative disease”.

The methods of the invention are also useful in the treatment of disorders associated with kidney disease. Therapeutic ABCB5+ stem cells previously injected into kidneys have been demonstrated to result in an almost immediate improvement in kidney function and cell renewal. Resnick, Mayer, Stem Cells Brings Fast Direct Improvement, Without Differentiation, in Acute Renal Failure, EurekAlert!, Aug. 15, 2005. Thus, the ABCB5+ stem cells of the invention may be administered to a subject having kidney disease alone or in combination with other therapeutics or procedures, such as dialysis, to improve kidney function and cell renewal.

Other diseases which may be treated according to the methods of the invention include diseases of the cornea and lung. Therapies based on the administration of therapeutic ABCB5+ stem cells in these tissues have demonstrated positive results. For instance, human therapeutic ABCB5+ stem cells have been used to reconstruct damaged corneas. Ma Y et al, Stem Cells, Aug. 18, 2005. Additionally stem cells derived from bone marrow were found to be important for lung repair and protection against lung injury. Rojas, Mauricio, et al., American Journal of Respiratory Cell and Molecular Biology, Vol. 33, pp. 145-152, May 12, 2005. Thus the ABCB5+ stem cells of the invention may also be used in the repair of corneal tissue or lung tissue.

Another use for the ABCB5+ stem cells of the invention is in tissue regeneration. In this aspect of the invention, the ABCB5 positive cells are used to generate tissue by induction of differentiation. Isolated and purified therapeutic ABCB5+ stem cells can be grown in an undifferentiated state through mitotic expansion in a specific medium and stored until they are ready for use. The cells are then thawed and activated to differentiate into bone, cartilage, and various other types of connective tissue by a number of factors, including mechanical, cellular, and biochemical stimuli. Human therapeutic ABCB5+ stem cells possess the potential to differentiate into cells such as osteoblasts and chondrocytes, which produce a wide variety of mesenchymal tissue cells, as well as tendon, ligament and dermis, and this potential is retained after isolation and for several population expansions in culture. Thus, by being able to isolate, purify, greatly multiply, and then activate therapeutic ABCB5+ stem cells to differentiate into the specific types of cells desired, such as skeletal and connective tissues such as bone, cartilage, tendon, ligament, muscle, and adipose, a process exists for treating skeletal and other connective tissue disorders. The term connective tissue is used herein to include the tissues of the body that support the specialized elements, and includes bone, cartilage, ligament, tendon, stroma, muscle and adipose tissue.

In another aspect, the present invention relates to a method for repairing connective tissue damage. The method comprises the steps of applying the stem cells to an area of connective tissue damage under conditions suitable for differentiating the cells into the type of connective tissue necessary for repair.

The term “connective tissue defects” refers to defects that include any damage or irregularity compared to normal connective tissue which may occur due to trauma, disease, age, birth defect, surgical intervention, etc. Connective tissue defects also refers to non-damaged areas in which bone formation is solely desired, for example, for cosmetic augmentation.

The single unit dose of ABCB5+ stem cells may be administered directly to a subject by any known mode of administration or may be seeded onto a matrix or implant in vitro (and then transferred in vivo) or directly in vivo. Matrices or implants include polymeric matrices such as fibrous or hydrogel based devices. Two types of matrices are commonly used to support the therapeutic ABCB5+ stem cells as they differentiate into cartilage or bone. One form of matrix is a polymeric mesh or sponge; the other is a polymeric hydrogel. Matrices may also be delivered to eye tissue.

The matrix may be biodegradeable or non-biodegradeable. The term biodegradable, as used herein, means a polymer that dissolves or degrades within a period that is acceptable in the desired application, less than about six months and most preferably less than about twelve weeks, once exposed to a physiological solution of pH 6-8 having a temperature of between about 25° C. and 38° C. A matrix may be biodegradable over a time period, for instance, of less than a year, more preferably less than six months, most preferably over two to ten weeks.

The cells may also be mixed with the hydrogel solution and injected directly into a site where it is desired to implant the cells, prior to hardening of the hydrogel. However, the matrix may also be molded and implanted in one or more different areas of the body to suit a particular application. This application is particularly relevant where a specific structural design is desired or where the area into which the cells are to be implanted lacks specific structure or support to facilitate growth and proliferation of the cells.

The site, or sites, where cells are to be implanted is determined based on individual need, as is the requisite number of cells. One could also apply an external mold to shape the injected solution. The suspension can be injected via a syringe and needle directly into a specific area wherever a bulking agent is desired, especially soft tissue defects.

As used herein, a subject is a human, non-human primate, cow, horse, pig, sheep, goat, dog, cat or rodent. Human ABCB5+ stem cells and human subjects are particularly important embodiments.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A device comprising
a unit of use vial (1) comprising 10.4×10⁶±20% to 15.6×10⁶ ABCB5+ stem cells per ml, wherein the unit of use vial (1) is a closed system cryogenic vial.

2. The device of claim 1, wherein the vial (1) comprises at least one fill tube (3) connected to a cell container (6).

3. The device of claim 1 or 2, wherein the vial (1) comprises a volume of 1-2 ml, 1.45-1.50 ml, 4-5 ml, 1-5 ml, or 4.05-4.96 ml.

4. The device of any one of claims 1-3, wherein the vial (1) comprises 13×10⁶+/−10% ABCB5+ stem cells per ml or 10.5×10⁶+/−10% cells/ml.

5. The device of any one of claims 1-4, a cell container (6), having a top end and a bottom end, wherein the top end is connected to an air vent (4) and a fill tube (3), wherein the air vent (4) and the fill tube (3) each have a distal surface and a proximal surface, and wherein the proximal surface of each is adjacent to the top end of the cell container (6).

6. The device of claim 5, wherein the fill tube (3) connects a fill port (2) to the top end of the cell container (6).

7. The device of claim 6, wherein the fill port (2) comprises a hermetically sealed fill attachment piece for attaching a cell delivery device (16).

8. The device of claim 7, wherein the fill attachment piece is a Luer lock.

9. The device of claim 5, wherein a microbiological filter (5) is positioned within the air vent (4).

10. A method for preparing a unit dose of a therapeutic cell solution, comprising preparing a pooled population comprising ABCB5-positive cells, concentrating the cells and resuspending the concentrated cells to produce a pooled sample having a cell concentration of 13×10⁶±20% cells/ml-24×10⁶/ml, optionally aliquoting a unit sample of cells from the pooled sample and transferring the unit sample to a fill tube (3) through a fill port (2) of a closed system cryogenic vial (1), and the unit sample is transferred into the cell container (6) to produce a unit dose of a therapeutic cell solution (7).

11. The method of claim 10, further comprising angling the vial (1) after the unit sample is transferred to the cell container (6) allowing a sterility testing sample to pass back into the fill tube (3), angling the vial (1) to an upright position and removing the sterility testing sample from the fill tube (3).

12. The method of claim 11, wherein the pooled sample has a cell concentration of about 13×10⁶+/−10% cells per ml.

13. The method of any one of claims 10-12, wherein the unit sample is about 2 ml or about 5 ml.

14. The method of any one of claims 11-13, wherein the vial (1) is angled at an about 90 degree angle.

15. The method of any one of claims 10-14, wherein the unit sample is transferred to the fill tube (3) using a Luer lock syringe (16) which is attached to the fill port (2).

16. The method of any one of claims 10-15, wherein the fill port (2) comprises a hermetically sealed fill attachment piece for attaching a cell delivery device (16).

17. The method of claim 11, wherein the sterility testing sample is removed from the fill tube (3) using a syringe.

18. The method of any one of claims 10-17, wherein the unit dose of a therapeutic cell solution (7) in the vial (1) comprises a volume of about at least 1.5 ml or 4.50 ml.

19. The method of any one of claims 10-18, wherein the unit dose of a therapeutic cell solution (7) in the vial (1) comprises about 13×10⁶±20% ABCB5+ stem cells per ml.

20. The method of any one of claims 10-19, further comprising thawing the vial of cells, removing the cells from the vial and reconstituting the cells in a syringe.

21. The method of any one of claims 10-19, further comprising thawing the vial of cells, removing the cells from the vial and reconstituting the cells in an infusion bag.

22. A method for treating a subject, comprising, administering to the subject a unit dose of a therapeutic cell solution, wherein the unit dose of a therapeutic cell solution is obtained from a device of any one of claims 1-9 or made according to a method of any one of claims 10-21.

23. The method of claim 22, wherein the cell solution is administered to a wound of the subject.

24. The method of claim 22 or 23, wherein multiple unit doses of therapeutic cell solution are administered to the subject.
